(19) United States
(12) Patent Application Publication
(10) Pub. No.: US 2012/0263803 A1
(43) Pub. Date: Oct. 18, 2012

(54) AQUEOUS OPHTHALMIC COMPOSITION

(75) Inventors: Yukihiko MASHIMA, Tokyo (JP); Yashuhiro HARADA, Tokyo (JP); Junichi KAWASAKI, Tokyo (JP); Ryuji UENO, Potomac, MD (US)

(73) Assignee: R-TECH UENO, LTD., Tokyo (JP)

(21) Appl. No.: 13/444,204

(22) Filed: Apr. 11, 2012

Related U.S. Application Data
(60) Provisional application No. 61/474,531, filed on Apr. 12, 2011.

(51) Int. Cl. A61K 33/22; A61P 27/06; A61P 27/02

(52) U.S. Cl. 424/659

(57) ABSTRACT

Disclosed is an aqueous ophthalmic composition comprising (a) a fatty acid derivative such as a prostaglandin derivative, (b) a polyoxyethylene sorbitan fatty acid ester, (c) an edetic acid compound, (d) a boric acid and a salt of a boric acid, (e) a pharmaceutically acceptable aqueous carrier, and (f) no more than 0.005 w/v % of benzalkonium chloride. The composition is stable and has good anti-microbial properties.

### CROSS REFERENCE TO RELATED APPLICATION

This application claims the benefit of U.S. Provisional Application No. 61/474,531 filed Apr. 12, 2011. The contents of this provisional application is herein incorporated by reference.

### TECHNICAL FIELD

The present invention relates to an aqueous ophthalmic composition that can be stored for long term in the manner that a specific fatty acid derivative comprised in the composition is kept stable. The present invention provides an aqueous ophthalmic composition comprising a specific fatty acid derivative and having enough anti-microbial properties even if the composition contains no or a very small amount of preservative such as benzalkonium chloride.

### BACKGROUND ART

Fatty acid derivatives are members of class of organic carboxylic acids, which are contained in tissues or organs of human and other mammals, and exhibit a wide range of physiological activities. Some fatty acid derivatives found in nature have, as a general structural property thereof, a prostanoic acid skeleton as shown in the formula (A):

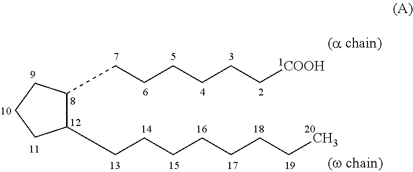

On the other hand, some synthetic Prostaglandin (PG) analogues have modified skeletons. The primary PGs are classified into PGAs, PGBs, PGCs, PGDs, PGEs, PGFs, PGGs, PGHs, PGIs and PGJs on the basis of the structural property of the five membered ring moiety, and are further classified into the following three types by the number and position of the unsaturated bond(s) in the carbon chain moieties.

Type 1 (subscript 1): 13,14-unsaturated-15-OH
Type 2 (subscript 2): 5,6- and 13,14-diunsaturated-15-OH
Type 3 (subscript 3): 5,6-, 13,14-, and 17,18-triunsaturated-15-OH.

Further, PGFs are classified on the basis of the configuration of the hydroxy group at the 9-position into a type (wherein the hydroxy group is of the α-configuration) and β type (wherein the hydroxy group is of the β-configuration).

Prostones, having an oxo group at position 15 of the prostanoic acid skeleton (15-keto type) and having a single bond between positions 13 and 14 and an oxo group at position 15 (13,14-dihydro-15-keto type)), have been known as substances naturally produced by enzymatic actions during metabolism of the primary PGs and have some therapeutic effect. Prostones have been disclosed in U.S. Pat. Nos. 5,073,569, 5,534,547, 5,225,439, 5,166,174, 5,428,062 5,380,709 5,886,034 6,265,440, 5,106,869, 5,221,763, 5,591,887, 5,770,759 and 5,739,161, the contents of these references are herein incorporated by reference.

Some fatty acid derivatives have been known as drugs used in the ophthalmic field, for example, for lowering intraocular pressure or treating glaucoma. For example, Isopropyl(Z)-7-[(1R,2R,3R,5S)-3,5-dihydroxy-2-[(3R)-3-hydroxy-5-phenylpentyl]cyclopentyl]-5-heptenoate (general name: latanoprost), Isopropyl (5Z)-7-((1R,2R,3R,5S)-3,5-dihydroxy-2-{(1E,3R)-3-hydroxy-4-[3-(trifluoromethyl)phenoxy]but-1-enyl}cyclopentyl)hept-5-enoate (general name: travoprost), (5Z)-7-{(1R,2R,3R,5S)-3,5-Dihydroxy-2-[(1E,3S)-3-hydroxy-5-phenylpent-1-en-1-yl]cyclopentyl}-N-ethylhept-5-enamide (general name: bimatoprost) and 1-Methylethyl(5Z)-7-[(1R,2R,3R,5S)-2-[(1E)-3,3-difluoro-4-phenoxy-1-butenyl]-3,5-dihydroxy cyclopentyl]-5-heptenoate (general name: tafluprost) have been marketed as ophthalmic solution for the treatment of glaucoma and/or ocular hypertension under the name of Xalatan®, Travatan®, Lumigan® and Tapros®, respectively.

Further, prostones have also been known to be useful in the ophthalmic field, for example, for lowering intraocular pressure and treating glaucoma (see U.S. Pat. Nos. 5,001,153, 5,151,444, 5,166,178, 5,194,429 and 5,236,907), for treating cataract (see U.S. Pat. Nos. 5,212,324 and 5,686,487), for increasing the choroidal blood flow (see U.S. Pat. No. 5,221,690), for treating optic nerve disorder (see U.S. Pat. No. 5,773,471), the contents of these references are herein incorporated by reference. Documents cited in this paragraph are herein incorporated by reference. Ophthalmic solution comprising (+)-isopropyl(Z)-7-[(1R,2R,3R,5S)-3,5-dihydroxy-2-(3-oxodecyl)cyclopentyl]hept-5-enoate (general name: isopropyl unoprostone) has been marketed under the name of Rescula® as a pharmaceutical product for the treatment of glaucoma and ocular hypertension.

In general, medicaments in the ophthalmic filed may preferably be formulated in an aqueous formulation suitable for topical ocular administration such as eye drops. Fatty acid derivatives are in general highly fat soluble and therefore, aqueous formulations comprising a fatty acid derivative need to be supplemented with a solublizing agent such as surface active agent. For example, isopropyl unoprostone can be formulated into an efficient aqueous ophthalmic composition effectively by using a polyoxyethylene sorbitan fatty acid ester such as polyoxyethylene sorbitan monooleate (polysorbate 80) (U.S. Pat. No. 5,236,907, the contents of the cited document is herein incorporated by reference).

Ophthalmic products such as eye drops that are provided with a multi-dose container and are stored for long term generally be supplemented with a preservative in order to have enough antimicrobial property. Benzalkonium chloride, a conventionally used preservative for manufacturing eye drops, has been reported to induce corneal epithelium disorder. It has, therefore, been desired to develop ophthalmic solutions that contain reduced amount of the preservatives as well as preservative free ophthalmic solutions that contain no preservatives such as benzalkonium chloride. For example, in a pharmaceutical composition comprising a fatty acid derivative, a sugar alcohol and a polyol such as glycerine, the amount of benzalkonium chloride can be reduced with keeping sufficient antimicrobial properties (WO2010/041722).

### SUMMARY OF THE INVENTION

An object of the present invention is to provide an aqueous ophthalmic composition that can be stored with keeping a specific fatty acid derivative stably for long term. Another object of the present invention to provides to an aqueous ophthalmic composition comprising the fatty acid derivative having enough antimicrobial properties even if the composition contains no or a very small amount of preservative such as benzalkonium chloride.

The inventors had found that an aqueous ophthalmic composition prepared by supplementing an edetic acid compound, a boric acid and a salt of a boric acid into an aqueous ophthalmic composition comprising a specific fatty acid derivative and a polyoxyethylene sorbitan fatty acid ester may have enough antimicrobial properties even if the composition contains only a very small amount of preservative such as benzalkonium chloride and can be stably stored with keeping the activity of the active ingredient for long term.

Accordingly, the present invention are as follows:

(1) An aqueous ophthalmic composition comprising:
(a) a fatty acid derivative represented by the formula (I);

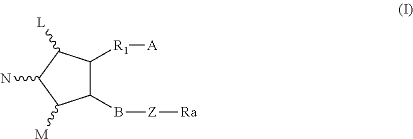

wherein L, M and N are hydrogen, hydroxy, halogen, lower alkyl, hydroxy(lower)alkyl, lower alkanoyloxy or oxo, wherein at least one of L and M is a group other than hydrogen and the five-membered ring may have at least one double bond;

A is —CH₃, —CH₂OH, —COCH₂OH, —COOH or a functional derivative thereof;
B is single bond, —CH₂—CH₂—, —CH═CH—, —C≡C—, —CH₂—CH₂—CH₂—, —CH═CH—CH₂—, —CH₂—CH═CH—, —C═C—CH₂— or —CH₂—C≡C—;

### Z is

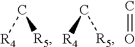

or single bond
wherein, R₄ and R₅ are hydrogen, hydroxy, halogen, lower alkyl, lower alkoxy or hydroxy(lower)alkyl, with the proviso that R₄ and R₅ are not hydroxy and lower alkoxy at the same time,

R_{L} is saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; or heterocyclic-oxy group, and at least one of carbon atom in the aliphatic hydrocarbon is optionally substituted by oxygen, nitrogen or sulfur; (b) a polyoxyethylene sorbitan fatty acid ester,

(c) an edetic acid compound,
(d) a boric acid and a salt of boric acid
(e) a pharmaceutically acceptable aqueous carrier, and
(f) no more than 0.005 w/v % of benzalkonium chloride.
(2) The composition of (1), wherein the amount of benzalkonium chloride in the composition is no more than 0.001 w/v %.
(3) The composition of (1), which comprises no benzalkonium chloride.
(4) The composition of (1), which comprises no preservative.
(5) The composition of (1), wherein B is —CH₂—CH₂— and Z is ═O.
(6) The composition of any one of (1)-(4) (2), wherein the fatty acid derivative is isopropyl unoprostone.
(7) The composition of any one of (1)-(6), wherein the polyoxyethylene sorbitan fatty acid ester is polyoxyethylene sorbitan monooleate.
(8) The composition of any one of (1)-(7), wherein the edetic acid compound is disodium edetate and its hydrate.
(9) The composition of any one of (1)-(8), wherein the boric acid is orthoboric and the salt of a boric acid is borax.
(10) The composition of any one of (1)-(9), wherein the aqueous pharmaceutically acceptable carrier is water.
(11) The composition of any one of (1)-(10), which is formulated as eye drops.
(12) The composition of (11), which is provided as a sterilized unit dose preparation.
(13) The composition of (12), which is provided as a daily unit dose preparation.
(14) The composition of (12), which is provided as a single unit dose preparation.
(15) The composition of (11), which is provided as a multi dose preparation.
(16) The composition of (15), which comprises no preservative.
(17) The composition of any one of (1)-(16), wherein the composition further comprises paraoxybenzoates.
(18) The composition of (17), wherein the paraoxybenzoates is methyl paraoxybenzoates and/or propyl paraoxybenzoates.
(19) The composition of any one of (1)-(18), wherein the composition further comprises sorbic acid and/or a salt of sorbic acid.
(20) The composition of (19), wherein the composition comprises sorbic acid.
(21) The composition of any one of (1)-(20), which is used for the treatment of a retinal disease or glaucoma and/or ocular hypertension.
(22) The composition of any one of (1)(4), (7)-(21), wherein the fatty acid derivative is latanoprost.
(23) The composition of any one of (1)-(21), which comprises in water:

0.15 w/v % or 0.12 w/v % of isopropyl unoprostone;

0.01-0.09 w/v % of disodium edetate dehydrate;

0.8-1.2 w/v % of polysorbate 80;

1.5-2 w/v % of orthoboric acid; and

borax in an amount to adjust the pH of the composition to 5.8-6.2.

(24) The composition of (23), wherein the amount of disodium edetate dehydrate is 0.01-0.03 w/v %.
(25) A method for the treatment of a retinal disease or glaucoma and/or ocular hypertension, which comprises administering to a subject in need thereof an aqueous ophthalmic composition of any one of (1)-(24).

The nomenclature of PG compounds used herein is based on the numbering system of prostanoic acid represented in the above formula (A).

The formula (A) shows a basic skeleton of the C-20 prostaglandin (PG) compound, but the present invention is not limited to those having the same number of carbon atoms. In the formula (A), the numbering of the carbon atoms which constitute the basic skeleton of the PG compounds starts at the carboxylic acid (numbered 1), and carbon atoms in the α-chain are numbered 2 to 7 towards the five-membered ring, those in the ring are 8 to 12, and those in the ω-chain are 13 to 20. When the number of carbon atoms is decreased in the α-chain, the number is deleted in the order starting from position 2; and when the number of carbon atoms is increased in the α-chain, compounds are named as substitution compounds having respective substituents at position 2 in place of carboxy group (C-1). Similarly, when the number of carbon atoms is decreased in the ω-chain, the number is deleted in the order starting from position 20; and when the number of carbon atoms is increased in the ω-chain, the carbon atoms at the position 21 or later are named as a substituent at position 20. Stereochemistry of the compounds is the same as that of the above formula (A) unless otherwise specified.

In general, each of PGD, PGE and PGF represents a PG compound having hydroxy groups at positions 9 and/or 11, but in the present specification they also include those having substituents other than the hydroxy groups at positions 9 and/or 11. Such compounds are referred to as 9-deoxy-9-substituted-PG compounds or 11-deoxy-11-substituted-PG compounds. A PG compound having hydrogen in place of the hydroxy group is simply named as 9- or 11-deoxy compound.

As stated above, the nomenclature of PG compounds is based on the prostanoic acid skeleton. In the case the compound has similar partial structure as the primary prostaglandin compound, the abbreviation of “PG” may be used. Thus, a PG compound whose α-chain is extended by two carbon atoms, that is, having 9 carbon atoms in the α-chain is named as 2-decarboxy-2-(2-carboxyethyl)-PG compound. Similarly, a PG compound having 11 carbon atoms in the α-chain is named as 2-decarboxy-2-(4-carboxybutyl)-PG compound. Further, PG compound whose ω-chain is extended by two carbon atoms, that is, having 10 carbon atoms in the ω-chain is named as 20-ethyl-PG compound. These compounds, however, may also be named according to the IUPAC nomenclatures.

The fatty acid derivative used in the present invention may be any substitution compound or derivative of the prostaglandin compound of formula (I), or formula (II) or formula (III) shown below. The PG derivative may be, for example, those having one double bond between positions 13 and 14, and a hydroxy group at position 15, those having one additional double bound between positions 5 and 6, those having a further double bond between positions 17 and 18. In addition, a 15-keto-PG compound having oxo group at position 15 instead of the hydroxy group; 15-deoxy PG compound having hydrogen instead of the hydroxy group at position 15; and a 15-fluoro PG compound having a fluorine at position 15 instead of the hydroxy group may also be included. Further, 13,14-dihydro compound in which the double bond between positions 13 and 14 is single bond and 13,14-didehydro-PG compound in which the double bond between the positions of 13 and 14 is triple bond may also be included. Further more, examples of the analogues including substitution compounds or derivatives of the PG compound include a PG compound whose carboxy group at the end of the α chain is esterified or amidated, or a physiologically acceptable salt thereof; a PG compound whose α or ω chain is shortened or extended than that of the primary PG; a PG compound having a side chain that having, for example 1-3 carbon atoms, on their α or ω chain; a PG compound having a substituent such as hydroxy, halogen, lower alkyl, hydroxy(lower)alkyl or oxo, or a double bond on its five membered ring; a PG compound having a substituent such as halogen, oxo, aryl and heterocyclic group on its α chain; a PG compound having a substituent such as halogen, oxo, hydroxy, lower alkoxy, lower alkanoyloxy, cyclo(lower)alkyl, cyclo(lower)alkyloxy, aryl, aryloxy, heterocyclic or heterocyclic-oxy on its ω chain; and a PG compound having shorter ω chain than that of normal prostanoic acid and having a substituent such as lower alkoxy, lower alkanoyloxy, cyclo(lower)alkyl, cyclo(lower)alkyloxy, aryl, aryloxy, heterocyclic or heterocyclic-oxy group at the end of the ω chain.

A preferred fatty acid derivative used in the present invention is represented by the formula (I):

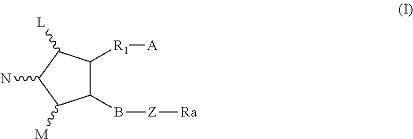

wherein L, M and N are hydrogen, hydroxy, halogen, lower alkyl, hydroxy(lower)alkyl, lower alkanoyloxy or oxo, wherein at least one of L and M is a group other than hydrogen and the five-membered ring may have at least one double bond;

A is —CH₃, —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C═C—CH₂— or —CH₂—C≡C—;

Z is

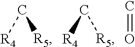

or single bond
wherein, R₄ and R₅ are hydrogen, hydroxy, halogen, lower alkyl, lower alkoxy or hydroxy(lower)alkyl, with the proviso that R₄ and R₅ are not hydroxy and lower alkoxy at the same time,

R₁ is saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is saturated or unsaturated lower or medium aliphatic hydrocarbon, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; or heterocyclic-oxy group and at least one of carbon atom in the aliphatic hydrocarbon is optionally substituted by oxygen, nitrogen or sulfur.

A more preferred fatty acid derivative used in the present invention is represented by the formula (II):

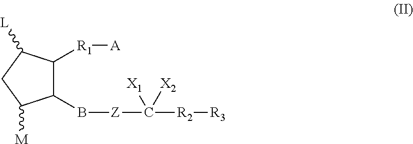

wherein L and N are hydrogen, hydroxy, halogen, lower alkyl, hydroxy(lower)alkyl or oxo, wherein at least one of L and M is a group other than hydrogen, and the five-membered ring may have at least one double bond;

A is —CH₃, —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

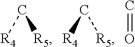

or single bond

wherein, R₄ and R₅ are hydrogen, hydroxy, halogen, lower alkyl, lower alkoxy or hydroxy (lower) alkyl, with the proviso that R₄ and R₅ are not hydroxy or lower alkoxy at the same time

X₁ and X₂ are hydrogen, lower alkyl, or halogen;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur;

R₂ is single bond or lower alkylene; and

R₃ is lower alkyl, lower alkoxy, cyclo(lower)alkyl, cyclo(lower)alkyloxy, aryl, aryloxy, heterocyclic group or heterocyclic-oxy group.

### PREFERRED EMBODIMENT TO CARRY OUT THE INVENTION

In the above formula (I), the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 6 to 10 carbon atoms for R₁ and 1 to 10, especially 1 to 8 carbon atoms for Ra.

The term “halogen atom” covers fluorine, chlorine, bromine and iodine.

The term “lower” is intended to include a group having 1 to 6 carbon atoms unless otherwise specified.

The term “lower alkyl” refers to a straight or branched chain saturated hydrocarbon group containing 1 to 6 carbon atoms and includes, for example, methyl, ethyl, propyl, isopropyl, butyl, isobutyl, t-butyl, pentyl and hexyl.

The term “lower alkylene” refers to a straight or branched chain bivalent saturated hydrocarbon group containing 1 to 6 carbon atoms and includes, for example, methylene, ethylene, propylene, isopropylene, butylene, isobutylene, t-butylene, pentylene and hexylene.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-1-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O—, wherein cyclo(lower)alkyl is as defined above.

The term “aryl” may include unsubstituted or substituted aromatic hydrocarbon rings (preferably monocyclic groups), for example, phenyl, tolyl, xylyl. Examples of the substituents are halogen and lower alkyl substituted by halogen, wherein halogen and lower alkyl are as defined above

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 types of hetero atoms selected from nitrogen atom, oxygen atom and, sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl and phenothiazinyl. Examples of the substituent in this case include halogen, and lower alkyl substituted by halogen, wherein halogen and lower alkyl group are as described above.

The term “heterocyclic-oxy group” means a group represented by the formula HcO—, wherein Hc is heterocyclic group as described above.

The term “functional derivative” of A includes salts, preferably pharmaceutically acceptable salts, ethers, esters and amides.

Suitable “pharmaceutically acceptable salts” include salts formed with non-toxic bases conventionally used in pharmaceutical field, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt including such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers; for example, lower alkyl ethers such as methyl ether, ethyl ether, propyl ether, isopropyl ether, butyl ether, isobutyl ether, sec-butyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and l-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A means a group represented by the formula —CONR′R″, wherein each of R′ and R″ is hydrogen, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M are hydrogen, hydroxy and oxo, and especially, L is hydroxy and M is hydroxy.

Preferred examples of A are —COOH and its pharmaceutically acceptable salt, ester and amide.

Preferred example of B is —CH₂—CH₂—.

Preferred example of X₁ and X₂ is hydrogen or halogen, more preferably, both of them are hydrogen or fluorine.

Preferred Z is C═O, or

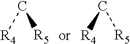

wherein one of R₄ and R₅ hydrogen and the other is hydroxy, and more preferably, Z is ═O that provides so called 15-keto type prostaglandin.

Preferred R₁ is an aliphatic hydrocarbon having 1-10 carbon atoms and more preferably, having 6-10 carbon atoms. Further, at least one carbon atom in the aliphatic hydrocarbon is optionally substituted by oxygen, nitrogen or sulfur.

Examples of R₁ may include, for example, the followings:

—CH₂—CH₂—CH₂—CH₂—CH₂—CH₂—,

—CH₂—CH═CH—CH₂—CH₂—CH₂—,

—CH₂—CH₂—CH₂—CH₂—CH═CH—,

—CH₂—C≡C—CH₂—CH₂—CH₂—,

—CH₂—CH₂—CH₂—CH₂—O—CH₂—,

—CH₂—CH═CH—CH₂—OβCH₂—,

—CH₂—C≡C—CH₂—O—CH₂—,

—CH₂—CH₂—CH₂—CH₂—CH₂—CH₂—CH₂—,

—CH₂—CH═CH—CH₂—CH₂—CH₂—CH₂—,

—CH₂—CH₂—CH₂—CH₂—CH₂—CH═CH—,

CH₂—C≡C—CH₂—CH₂—CH₂—CH₂—,

—CH₂—CH₂—CH₂—CH₂—CH₂—CH(CH₃)—CH₂—,

—CH₂—CH₂—CH₂—CH₂—CH(CH₃)—CH₂—,

—CH₂—CH₂—CH₂—CH₂—CH₂—CH₂—CH₂—CH₂—,

—CH₂—CH═CH—CH₂—CH₂—CH₂—CH₂—CH₂—,

—CH₂—CH₂—CH₂—CH₂—CH₂—CH₂—CH═CH₂—,

—CH₂—C≡C—CH₂—CH₂—CH₂—CH₂—CH₂—, and

—CH₂—CH₂—CH₂—CH₂—CH₂—CH₂—CH(CH₃)—CH₂—.

Preferred Ra is a hydrocarbon containing 1-10 carbon atoms, more preferably, 1-8 carbon atoms. Ra may have one or two side chains each having one carbon atom.

Preferred R₂ is single bond.

Preferred R₃ is a lower alkyl, aryl or aryloxy. Especially lower alkyl having 4-6 carbon atoms, phenyl or phenyloxy. R₃ may have one or two side chains each having one carbon atom.

The configuration of the ring and the α- and/or ω chains, in the above formulae (I) and (II) may be the same as or different from that of the primary PGs. The present invention also includes a mixture of a compound having the primary type configuration and a compound of a non-primary type configuration.

The typical examples of the compounds used in the present invention are (Z)-7-[(1R,2R,3R,5S)-3,5-dihydroxy-2-(3-oxodecyl)cyclopentyl]hept-5-enoic acid, Isopropyl(Z)-7-[(1R,2R,3R,5S)-3,5-dihydroxy-2-[(3R)-3-hydroxy-5-phenylpentyl]cyclopentyl]-5-heptenoic and derivatives and analogs thereof. The most preferable compound in the present invention is (+)-isopropyl(Z)-7-[(1R,2R,3R,5S)-3,5-dihydroxy-2-(3-oxodecyl)cyclopentyl]hept-5-enoate, hereafter, this compound may be called as isopropyl unoprostone.

In one embodiment, a fatty acid derivative wherein the bond between the positions of 13 and 14 is single bond may be in the keto-hemiacetal equilibrium by formation of a hemiacetal between hydroxy at position 11′ and keto at position 15.

It has been revealed that when both of X₁ and X₂ are halogen atoms, especially, fluorine atoms, the compound contains a tautomeric isomer, bicyclic compound.

If such tautomeric isomers as above are present, the proportion of both tautomeric isomers varies with the structure of the rest of the molecule or the kind of the substituent present. Sometimes one isomer may predominantly be present in comparison with the other. The fatty acid derivative in this embodiment includes both isomers.

In this embodiment, the fatty acid derivative may further include the bicyclic compound and analogs or derivatives thereof.

The bicyclic compound is represented by the formula (III):

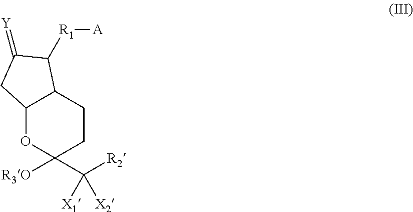

wherein, A is —CH₃, —CH₂OH, —COCH₂OH, —COOH or a functional derivative thereof;

X₁′ and X₂′ are hydrogen, lower alkyl, or halogen;

Y is

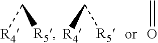

wherein R₄′ and R₅′ are hydrogen, hydroxy, halogen, lower alkyl, lower alkoxy or hydroxy(lower)alkyl, wherein R₄′ and R₅′ are not hydroxy and lower alkoxy at the same time.

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one carbon atom in the aliphatic hydrocarbon is optionally substituted by oxygen, nitrogen or sulfur;

R₂′ is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group and at least one of carbon atom in the aliphatic hydrocarbon is optionally substituted by oxygen, nitrogen or sulfur; and

R₃′ is hydrogen, lower alkyl, cyclo(lower)alkyl, aryl or heterocyclic group.

While the compounds used in this embodiment may be represented by a formula or name based on the keto-type compound regardless of the presence or absence of the isomers, it is to be noted that such structure or name does not intend to exclude the hemi-acetal type compound.

In the present invention, any of isomers such as the individual tautomeric isomers, the mixture thereof, or optical isomers, the mixture thereof, a racemic mixture, and other steric isomers may be used in the same purpose.

Some of the compounds used in the present invention may be prepared by the method disclosed in U.S. Pat. Nos. 5,073,569, 5,166,174, 5,221,763, 5,212,324, 5,739,161 and 6,242,485, the contents of these references are herein incorporated by reference.

Some of the fatty acid derivatives shown in this specification are useful for manufacturing ophthalmic composition for various uses. Especially, ophthalmic compositions useful for the treatment of glaucoma and/or ocular hypertension, central chorioretinopathy, central chorioretinopathy, hypertensive retinopathy, age-related macular degeneration, arteriosclerotic retinopathy, renal retinopathy, retinopathy diabetic, retinal artery occlusion, retinal vein occlusion, retinal detachment, macular edema, retinitis pigmentosa, prematurity, anemic retinopathy, leukemic retinopathy, retinal/choroidal disorders due to external injury, optic neuritis, papilloretinitis, papillitis, neuroretinitis, arachnitis, myelitis, optic nerve atrophy (including diseases associated with optic nerve atrophy, such as Leber's hereditary optic neuropathy (including Lever's disease), optic ischaemic neuropathy, idiopathic optic neuritis, glaucomatous optic neuropathy, optic nerve trauma and others), ocular neovascularization such as choroidal neovascularization and retinal neovascularization, or other retinal diseases such as eyeground diseases can be manufactured.

The term “treatment” or “treating” used herein refers to any means of control of a condition including prevention, cure, relief of the condition, attenuation of the condition and arrest of progression.

In the pharmaceutical composition of the present invention, the fatty acid derivative, the active ingredient, may be the above described compound.

The amount of the fatty acid derivative in the ophthalmic composition may be determined suitably depending on the compound used, type, age, weight of the subject to be treated, condition to be treated, desired effect of the treatment, the volume to be administered and the term for the treatment.

The ophthalmic composition of the present invention is an aqueous ophthalmic formulation that comprises the fatty acid derivative as an active ingredient and may be provided as eye drops. The amount of the fatty acid derivative contained in the ophthalmic composition of the present invention may be about 0.0001-10 w/v %, preferably, 0.0001-5 w/v % and more preferably, 0.001-1 w/v %.

In the case the fatty acid derivative is isopropyl unoprostone, the amount of isopropyl unoprostone in the aqueous ophthalmic composition is preferably about 0.12 or about 0.15 w/v %.

In one embodiment, the ophthalmic composition may be provided as a sterile unit dose preparation. Examples of the sterile unit dose preparations may be a daily unit dose preparation that can be used for one day only for plural instillation to the eyes and a single unit dose preparation that can be used for single instillation only. In another embodiment, the ophthalmic composition may be provided as a multi-dose preparation that can be instilled repeatedly for plural days, for example, up to 30 days after opening the preparation.

Examples of the polyoxyethylene sorbitan fatty acid esters may include polyoxyethylene sorbitan monooleate (Polysorbate 80), polyoxyethylene sorbitan monostearate (Polysorbate 60), polyoxyethylene sorbitan monopalmitate (Polysorbate 40), polyoxyethylene sorbitan monolaurate, polyoxyethylene sorbitan trioleate and polyoxyethylene sorbitan tristearate (Polysorbate 65). Polyoxyethylene sorbitan monooleate (Polysorbate 80) is preferably used. The amount of the polyoxyethylene sorbitan tatty acid ester in the ophthalmic composition may be about 0.01-5 w/v %, preferably, about 0.05-2 w/v % and more preferably, 0.5-1.5 w/v %.

“Edetic acid compound” in this specification and claims represents a compound selected from edetic acid (ethylene diamine tetra-acetic acid), a salt thereof or a chilate of the acid and 1-4 valents metal ion, and a hydrorate thereof. Examples of edetic acid compounds may include edetic acid, monosodium edetate, disodium edetate, trisodium edetate, tetrasodium edetate, calcium disodium edetate, dipoptassium edetate, disodium edetate dihydrate, tetrasodium edetate dihydrate, tetrasodium edetate tetrahydrate. Disodium edetate and its hydrates are preferably used. The amount of the edetic acid compound in the ophthalmic composition may be about 0.001-1 w/v % in general and preferably, about 0.01-0.5 w/v % and more preferably, about 0.01-0.09 w/v %. In more detail, for single unit dose preparation that is used for single instillation only, the amount of the edetic acid compound in the ophthalmic composition may be about 0.01-0.09 w/v %. For multi-dose preparation, the amount of the edetic acid compound in the composition may preferably be about 0.001-0.05 w/v %, more preferably, about 0.01-0.03 w/v %.

“Boric acid” in the specification and claims may be not only orthoboric acid but also polyboric acid such as metaboric acid and diboric acid. The amount of boric acid in the ophthalmic composition of the present invention may be about 0.5-2.0 w/v %, preferably, about 1.0-2.0 w/v % and more preferably, about 1.5-2.0 w/v %.

“Salt of a boric acid” may be any salt generated by the neutralization of a boric acid with a base, and may be, for example, a salt of orthoboric acid, a salt of diboric acid, a salt of metaboric acid, and a salt of tetraboric acid such as borax. Borax is preferable. The salt of a boric acid is added to the ophthalmic composition so that pH of the composition is about 6, i.e. pH 5.5-6.5, more preferably, pH 5.8-6.2.

In the specification and claims, the pharmaceutically acceptable aqueous carrier may be any material that can dissolve or disperse the fatty acid derivative. Water in the form of distilled water or physiologically acceptable saline is preferably employed.

According to the present invention, by adding an edetic acid compound, a boric acid and a salt of a boric to an aqueous composition containing the specific fatty acid derivative and polyoxyethylene sorbitan fatty acid ester, an aqueous composition that exert enough antimicrobial property even if the amount of the preservative such as benzalkonium chloride contained in the composition is very small and can keep the fatty acid derivative in the composition stably. In one embodiment, an aqueous ophthalmic composition comprising no more than 0.005 w/v %, and preferably, no more than 0.001 w/v % of benzalkonium chloride is provided. In another embodiment, benzalkonium chloride free and preservative free compositions are provided.

In the specification and claims, “preservative” represents a substance that is added to a product to prevent invasion, growth and proliferation of microorganisms so that the product does not corrupt or ferment. In the specification and claims, preservative should be a pharmaceutically acceptable preservative. Examples of preservatives may comprise, but not limited to, quaternary ammonium preservatives such as benzalkonium chloride and benzethonium chloride, benzoic acid derivatives such as benzoic acid and sodium benzoate, chlorohexidines such as gluconate chlorohexidine, paraoxybenzoic acid esters such as methyl paraoxybenzoates and propyl paraoxybenzoates, sorbic acid derivatives such as sorbic acid and potassium sorbate, alcohols such as chlorobutanol.

In one embodiment, the ophthalmic composition may contain a paraoxybenzoate, sorbic acid or its salt in the case higher antimicrobial property is required without affecting the stability of the fatty acid derivative.

Examples of paraoxybenzoic acid esters may include methyl, ethyl, propyl and butyl benzoates and a combination thereof. Preferably, methyl paraoxybenzoates and propyl paraoxybenzoates are used. The amount of the paraoxybenzoic acid ester in the composition may be about 0.0005-1 w/v %, preferably, about 0.001-5 w/v %.

Examples of sorbic acid derivatives may include sorbic acid and potassium sorbate, and sorbic acid is preferable. The sorbic acid derivative in the ophthalmic composition of the present invention may be about 0.005-10 w/v % and preferably, about 0.01-5 w/v %.

The ophthalmic composition of the present invention may further comprise an additive that has been employed in the field of ophthalmology. Examples of the additives may include thickeners, for example, polysaccharides such as sodium hyaluronate, chondroitin sulfate, guar gum, gellan gum, xantan gum and sodium alginate; cellulose polymers such as methyl cellulose, methyl ethyl cellulose and hydroxypropyl methyl cellulose; sodium polyacrylate; a carboxyvinyl polymer and a crosslinked polyacrylic acid; and may include buffering agents, for example, organic amines such as tromethamol or ethanol amine, organic acid salts such as citrate or lactate, and phosphoric acid.

In a preferable embodiment, an aqueous ophthalmic composition which comprises in water: 0.15 w/v % of isopropyl unoprostone; 0.01-0.07 w/v % of disodium edetate dehydrate; 0.8-1.2 w/v % of polysorbate 80; 1.5-2 w/v % of orthoboric acid and borax in an amount to adjust the pH of the composition to 5.8-6.2, is provided. In more preferable embodiment, an aqueous ophthalmic composition which comprises in water: 0.15 w/v % of isopropyl unoprostone; about 0.02 or 0.05 w/v %, especially, about 0.02 w/v % of disodium edetate dehydrate; about 1 w/v % of polysorbate 80; about 1.71-1.8 w/v % of orthoboric acid and borax in an amount to adjust the pH of the composition to 5.8-6.2, is provided. The latter composition may preferably used for manufacturing multi-dose preparations with good antimicrobial properties.

In another preferred embodiment, an aqueous ophthalmic composition which comprises in water: 0.12 w/v % of isopropyl unoprostone; 0.01-0.03 w/v % of disodium edetate dehydrate; 0.8-1.2 w/v % of polysorbate 80; 1.5-2 w/v % of orthoboric acid and borax in an amount to adjust the pH of the composition to 5.8-6.2, is provided. In more preferable embodiment, an aqueous ophthalmic composition which comprises in water: 0.12 w/v % of isopropyl unoprostone; about 0.02 w/v % of disodium edetate dehydrate; about 1 w/v % of polysorbate 80; about 1.71-1.9 w/v % of orthoboric acid and borax in an amount to adjust the pH of the composition to 5.8-6.2, is provided. The latter composition may preferably be used for manufacturing multi-dose preparations with good antimicrobial properties.

The present invention will be described in more detail with reference to the following examples, which is not intended to limit the scope of the present invention. In the following examples, “boric acid” refers “ortho-boric acid”.

### EXAMPLES

In the following formulation and test examples, “%” represents “w/v %” unless otherwise indicated.

### Formulation Example 1

The ingredients shown below were dissolved in purified water and the solution was aseptically filtered and then filled into a sterile unit dose vial (one-day disposable type) by a Blow Fill Seal system to give sterile one day unit dose type eye drops.

| | |
|---|---|
| 0.15% | isopropyl unoprostone |
| 1.0% | Polyoxyethylene sorbitan monooleate |
| 1.65% | boric acid |
| 0.02% | borax |
| 0.05% | disodium edetate dihydrate |

### Formulation Examples 2, 3 and 4

The ingredients shown below were dissolved in purified water and the solution was aseptically filtered and then filled into a sterile unit dose vial (one-day disposable type) by a Blow Fill Seal system to give sterile one day unit dose type eye drops.

| | |
|---|---|
| 0.15% | isopropyl unoprostone |
| 1.0% | Polyoxyethylene sorbitan monooleate |
| 1.65% | boric acid |
| 0.035% | borax |
| 0.05% | disodium edetate dihydrate |
| 0.2, 0.4 or 0.6% | gellan gum |

### Formulation Example 5

The ingredients shown below were dissolved in purified water and the solution was aseptically filtered and then filled into a sterile unit dose vial (one-day disposable type) by a Blow Fill Seal system to give sterile one day unit dose type eye drops.

| | |
|---|---|
| 0.15% | isopropyl unoprostone |
| 1.0% | Polyoxyethylene sorbitan monooleate. |
| 1.65% | boric acid |
| 0.02% | borax |
| 0.05% | disodium edetate dihydrate |
| 0.6% | xanthane gum |

### Formulation Example 6

The ingredients shown below were dissolved in purified water and the solution was aseptically filtered and then filled into a sterile unit dose vial (one-day disposable type) by a Blow Fill Seal system to give sterile one day unit dose type eye drops.

| | |
|---|---|
| 0.005% | latanoprost |
| 0.2% | Polyoxyethylene sorbitan monooleate |
| 1.72% | boric acid |
| 0.036% | borax |
| 0.1% | disodium edetate dihydrate |

### Formulation Example 7

The ingredients shown below were dissolved in purified water and the solution was filled into a sterilized low density polyethylene (LDPE) multi-dose bottle under sterile condition to give multi-dose type eye drops.

| | |
|---|---|
| 0.15% | isopropyl unoprostone |
| 1.0% | Polyoxyethylene sorbitan monooleate |
| 1.90% | boric acid |
| 0.03% | borax |
| 0.05% | disodium edetate dihydrate |
| 0.005% | benzalkonium choloride |

### Formulation Example 8

The ingredients shown below were dissolved in purified water and the solution was filled into sterilized low density polyethylene (LDPE) multi-dose bottle under sterile condition to give multi-dose type eye drops.

| | |
|---|---|
| 0.12% | isopropyl unoprostone |
| 1.0% | Polyoxyethylene sorbitan monooleate |
| 1.71% | boric acid |
| 0.02% | borax |
| 0.05% | disodium edetate dihydrate |
| 0.001% | benzalkonium choloride |

### Formulation Example 9

The ingredients shown below were dissolved in purified water and the solution was filled into a sterilized low density polyethylene (LDPE) multi-dose bottle under sterile condition to give multi-dose type eye drops.

| | |
|---|---|
| 0.12% | isopropyl unoprostone |
| 1.0% | Polyoxyethylene sorbitan monooleate |
| 1.71% | boric acid |
| 0.02% | borax |
| 0.02% | disodium edetate dihydrate |

### Test Example 1

The ingredients shown below were dissolved in purified water and the solution was aseptically filtered to give test solution 1.

| | |
|---|---|
| 0.15% | isopropyl unoprostone |
| 1.0% | polysorbate 80 |
| 1.71% | boric acid |
| 0.026% | borax |
| 0.1% | disodium edetate dihydrate |

In the same manner as test solution 1, test solution 2 containing the following ingredients in water was prepared.

| | |
|---|---|
| 0.15% | isopropyl unoprostone |
| 1.0% | polysorbate 80 |
| 1.9% | concentrated glycerine |
| 1.0% | D-mannitol |
| 0.1% | disodium edetate dihydrate |

Test solutions 1 and 2 were tested for preservatives-effectivencss tests according to the Japanese Pharmacopeia, 15th edition. The tests were conducted by using the following test microorganisms: Escherichia coli(E. coli, Pseudomonas aeruginosa(P. aeruginosa), Staphylococcus aureus(S. aureus), Aspergillus niger(A. niger) and Candida albicans(C. albicans). The sterilized test solutions 1 and 2 were respectively distributed into each of 5 separate containers, and each container was inoculated with a separate test microorganism (mentioned above). Inoculated test solutions were kept at 20-25° C. with protection from light, and sampled to determine microorganism concentration at after 7, 14 and 28 days from the inoculation. The microorganism count at each interval was compared to the inoculum count. Results are summarized in table 1 below. In the table, “Log reduction” represents Log(inoculum count/count at sampling). “N.D.” represents no detection and “N.S. represents the no increase.

TABLE 1

| | | Log reduction of | | |
|---|---|---|---|---|
| | | microorganism count | | |
| test | | After 7 | after 14 | after 28 |
| solutions | Microorganisms | days | days | days |
| 1 | E. coli | N.D. | N.D. | N.D. |
| | P. aeruginosa | N.D. | N.D. | N.D. |
| | S. aureus | N.D. | N.D. | N.D. |
| | A. niger | N.I. | N.I. | N.I. |
| | C. albicans | N.I. | N.I. | N.D. |
| 2 | E. coli | 2.2 | 4.3 | 5.2 |
| | P. aeruginosa | N.D. | N.D. | N.D. |
| | S. aureus | N.D. | N.D. | N.D. |
| | A. niger | N.I. | N.I. | N.I. |
| | C. albicans | N.I. | N.I. | N.I. |

As shown in the above result, the microorganism count in the test solution 1 that contains boric acid was significantly reduced from the inoculated count. This reduction was superior than that in the test solution 2 containing no boric acid. Although test solution 1 did not contain a preservative such as benzalkonium chloride, the solution had enough anti-microbial effectiveness.

### Test Example 2

Test Solution 3 containing the following ingredients in water was prepared in the same manner as test solution 1 in test example 1.

| | |
|---|---|
| 0.15% | isopropyl unoprostone |
| 1.0% | polysorbate 80 |
| 1.71% | boric acid |
| 0.026% | borax |
| 0.05% | disodium edetate dehydrate |

Test Solution 4 containing the following ingredients in water was prepared in the same manner as test solution 1 in test example 1.

| | |
|---|---|
| 0.15% | isopropyl unoprostone |
| 1.0% | polysorbate 80 |
| 1.71% | boric acid |
| 0.026% | borax |

Test solutions 3 and 4 were filled in sterile law-density polyethylene (LDPE) containers respectively. The container was kept at 55° C. for two weeks and the concentration of isopropyl unoprostone in the solution was determined by means of a liquid chromatograph. Results are shown in Table 2.

TABLE 2
Stability of isopropyl unoprostone(IU): Stored two weeks at 55° C.:

concentration of IU: %
vs. initial concentration

| | Initial | 2 weeks at 55° C. |
|---|---|---|
| test solution 3 | 100 | 99.6 |
| test solution 4 | 100 | 77.6 |

It is apparent from the table as above that test solution 4 that does not contain disodium edetate dihydrate could not maintain isopropyl unoprostone stably.

### Test Example 3

Test Solution 5 containing the following ingredients in water was prepared in the same manner as test solution 1 in test example 1.

| | |
|---|---|
| 0.005% | latanoprost |
| 0.2% | polysorbate 80 |
| 1.72% | boric acid |
| 0.036% | borax |
| 0.1% | disodium edetate dihydrate |
| 0.035% | methyl paraoxybenzoate |
| 0.003% | propyl paraoxybenzoate |

Test Solution 6 containing the following ingredients in water was prepared in the same manner as test solution 1 in test example 1.

| | |
|---|---|
| 0.005% | latanoprost |
| 0.2% | polysorbate 80 |
| 1.26% | boric acid |
| 0.27% | borax |
| 0.1% | disodium edetate dihydrate |
| 0.05% | sorbic acid |

Test solutions 5 and 6 were tested for preservative effectiveness test in the same manner as test example 1. The results are shown in Table 3.

TABLE 3

| | | Log reduction of | | |
|---|---|---|---|---|
| | | microorganism count | | |
| test | | After 7 | after 14 | after 28 |
| solutions | Microorganisms | days | days | days |
| 5 | E. coli | N.D. | N.D. | N.D. |
| | P. aeruginosa | N.D. | N.D. | N.D. |
| | S. aureus | N.D. | N.D. | N.D. |
| | A. niger | N.I. | N.I. | N.I. |
| | C. albicans | N.I. | N.I. | N.D. |
| 6 | E. coli | 1.8 | N.D. | N.D. |
| | P. aeruginosa | N.D. | N.D. | N.D. |
| | S. aureus | N.D. | N.D. | N.D. |
| | A. niger | N.I. | N.I. | N.I. |
| | C. albicans | N.I. | N.I. | N.I. |

Although they do not contain benzalkonium chloride, the test solutions 5 and 6 exhibited enough anti-microbial properties.

### Test Example 4

Results of the preservative effectiveness tests may be affected by the facility where the tests were conducted and the cell number of inoculated microorganisms. In order to evaluate reproducibility of the preservative effectiveness tests, test solutions shown in tables 4 and 6′ were tested for the preservative effectiveness tests according to the Japanese Pharmacopeia, 15th edition in three (3) different facilities. Sterile test solutions were prepared in the same manner as test solution 1 in test example 1. The test solutions were evaluated under criteria for Category IA product (sterile preparations). Results are summarized in Tables 5 and 7. In the table, “t” represents the test section that did not meet the criteria.

Criteria required under the Japanese Pharmacopeia, 15th edition for category IA products are as follows:

| | | Criteria | | |
|---|---|---|---|---|
| | | After 7 | After 14 | After 28 |
| microorganisms | | days | days | days |
| bacteria | E. coli | No | 0.1% of | Same or |
| | P. aeruginosa | criteria | inoculum | less than |
| | S. aureus | is | count or | level after |
| | | available | less. Here, | 14 days |
| | | | 3.0 Log | |
| | | | reduction | |
| | | | was | |
| | | | interpreted | |
| | | | as “0.1%”. | |
| fungi | A. niger | No | Same or less | Same or |
| | C. albicans | criteria | than | less than |
| | | is | inoculum | inoculum |
| | | available | count | count |

TABLE 4

| | | | | | pH | |
|---|---|---|---|---|---|---|
| Test | | poly- | disodium | | modi- | pH |
| Solu- | isopropyl | sorbate | edetate | boric | fier | measured |
| tions | unoprostone | 80 | dihydrate | acid | (borax) | value |
| 7 | 0.15% | 1% | 0.1% | 1.71% | 0.04% | 5.97 |
| 8 | 0.15% | 1% | 0.1% | 1.75% | 0.04% | 6.01 |
| 9 | 0.15% | 1% | 0.1% | 1.8% | 0.05% | 5.98 |
| 10 | 0.15% | 1% | 0.1% | 1.9% | 0.05% | 5.98 |

TABLE 5

| | | | initial | Log reduction | | |
|---|---|---|---|---|---|---|
| | | | inoculum | 7 | 14 | 28 |
| | facility | Microorganisms | count | days | days | days |
| test | A | E. coli | 310,000 | 3.6 | N.D. | N.D. |
| solution 7 | | P. aeruginosa | 140,000 | 3.2 | N.D. | N.D. |
| | | S. aureus | 390,000 | N.D. | N.D. | N.D. |
| | | A. niger | 190,000 | N.I. | N.I. | N.I. |
| | | C. albicans | 400,000 | N.I. | N.D. | N.D. |
| | B | E. coli | 390,000 | 1.3 | 2.8^{†} | N.D. |
| | | P. aeruginosa | 370,000 | N.D. | N.D. | N.D. |
| | | S. aureus | 180,000 | N.D. | N.D. | N.D. |
| | | A. niger | 300,000 | N.I. | N.I. | N.I. |
| | | C. albicans | 300,000 | N.I. | N.I. | N.D. |
| | C | E. coli | 340,000 | 1.0 | N.D. | N.D. |
| | | P. aeruginosa | 480,000 | N.D. | N.D. | N.D. |
| | | S. aureus | 350,000 | N.D. | N.D. | N.D. |
| | | A. niger | 300,000 | N.I. | N.I. | N.I. |
| | | C. albicans | 430,000 | N.I. | N.I. | N.D. |
| test | A | E. coli | 310,000 | 3.7 | N.D. | N.D. |
| solution 8 | | P. aeruginosa | 140,000 | 2.9 | 3.6 | N.D. |
| | | S. aureus | 390,000 | N.D. | N.D. | N.D. |
| | | A. niger | 190,000 | N.I. | N.I. | N.I. |
| | | C. albicans | 100,000 | N.I. | N.D. | N.I. |
| | B | E. coli | 390,000 | 1.1 | 2.6^{†} | N.D. |
| | | P. aeruginosa | 370,000 | N.D. | N.D. | N.D. |
| | | S. aureus | 180,000 | N.D. | N.D. | N.D. |
| | | A. niger | 300,000 | N.I. | N.I. | N.I. |
| | | C. albicans | 300,000 | N.I. | N.D. | N.D. |
| | C | E. coli | 340,000 | 1.3 | N.D. | N.D. |
| | | P. aeruginosa | 480,000 | N.D. | N.D. | N.D. |
| | | S. aureus | 350,000 | N.D. | N.D. | N.D. |
| | | A. niger | 300,000 | N.I. | N.I. | N.I. |
| | | C. albicans | 430,000 | N.I. | N.I. | N.D. |
| test | A | E. coli | 310,000 | 2.9 | N.D. | N.D. |
| solution 9 | | P. aeruginosa | 140,000 | N.D. | N.D. | N.D. |
| | | S. aureus | 390,000 | N.D. | N.D. | N.D. |
| | | A. niger | 190,000 | N.I. | N.I. | N.I. |
| | | C. albicans | 400,000 | N.I. | N.D. | N.D. |
| | B | E. coli | 390,000 | 1.9 | 4.0 | N.D. |
| | | P. aeruginosa | 370,000 | N.D. | N.D. | N.D. |
| | | S. aureus | 180,000 | N.D. | N.D. | N.D. |
| | | A. niger | 300,000 | N.I. | N.I. | N.I. |
| | | C. albicans | 300,000 | N.I. | N.I. | N.D. |
| | C | E. coli | 340,000 | 1.3 | N.D. | N.D. |
| | | P. aeruginosa | 480,000 | N.D. | N.D. | N.D. |
| | | S. aureus | 350,000 | N.D. | N.D. | N.D. |
| | | A. niger | 300,000 | N.I. | N.I. | −0.6^{†} |
| | | C. albicans | 430,000 | N.I. | N.I. | N.D. |
| test | A | E. coli | 310,000 | 2.5 | 4.3 | N.D. |
| solution | | P. aeruginosa | 140,000 | N.D. | N.D. | N.D. |
| 10 | | S. aureus | 390,000 | N.D. | N.D. | N.D. |
| | | A. niger | 190,000 | N.I. | N.I. | N.I. |
| | | C. albicans | 400,000 | N.I. | N.D. | N.D. |
| | B | E. coli | 390,000 | 1.3 | 3.3 | N.D. |
| | | P. aeruginosa | 370,000 | N.D. | N.D. | N.D. |
| | | S. aureus | 180,000 | N.D. | N.D. | N.D. |
| | | A. niger | 300,000 | N.I. | N.I. | N.I. |
| | | C. albicans | 300,000 | N.I. | N.I. | N.D. |
| | C | E. coli | 340,000 | 1.0 | N.D. | N.D. |
| | | P. aeruginosa | 480,000 | N.D. | N.D. | N.D. |
| | | S. aureus | 350,000 | N.D. | N.D. | N.D. |
| | | A. niger | 300,000 | N.I. | N.I. | N.I. |
| | | C. albicans | 430,000 | N.I. | N.I. | N.D. |

TABLE 6

| | | | | | pH | |
|---|---|---|---|---|---|---|
| Test | | poly- | disodium | | modi- | pH |
| Solu- | isopropyl | sorbate | edetate | boric | fier | measured |
| tions | unoprostone | 80 | dihydrate | acid | (borax) | value |
| 11 | 0.15% | 1% | 0.05% | 1.71% | 0.03% | 5.99 |
| 12 | 0.15% | 1% | 0.05% | 1.75% | 0.04% | 5.98 |
| 13 | 0.15% | 1% | 0.05% | 1.8% | 0.04% | 5.98 |
| 14 | 0.15% | 1% | 0.05% | 1.9% | 0.04% | 5.99 |

TABLE 7

| | | | initial | Log reduction | | |
|---|---|---|---|---|---|---|
| | | | inoculum | | 14 | 28 |
| | facility | Microorganisms | count | 7 days | days | days |
| test | A | E. coli | 310,000 | 3.4 | N.D. | N.D. |
| solution | | P. aeruginosa | 140,000 | N.D. | N.D. | N.D. |
| 11 | | S. aureus | 390,000 | N.D. | N.D. | N.D. |
| | | A. niger | 190,000 | N.I. | N.I. | N.I. |
| | | C. albicans | 400,000 | N.I. | N.D. | N.D. |
| | B | E. coli | 390,000 | 1.9 | 4.1 | N.D. |
| | | P. aeruginosa | 370,000 | N.D. | N.D. | N.D. |
| | | S. aureus | 180,000 | N.D. | N.D. | N.D. |
| | | A. niger | 300,000 | N.I. | N.I. | N.I. |
| | | C. albicans | 300,000 | N.I. | N.I. | N.D. |
| | C | E. coli | 340,000 | 0.9 | N.D. | N.D. |
| | | P. aeruginosa | 480,000 | N.D. | N.D. | N.D. |
| | | S. aureus | 350,000 | N.D. | N.D. | N.D. |
| | | A. niger | 300,000 | N.I. | N.I. | N.I. |
| | | C. albicans | 430,000 | N.I. | N.I. | N.D. |
| test | A | E. coli | 310,000 | 2.6 | N.D. | N.D. |
| solution | | P. aeruginosa | 140,000 | 4.0 | N.D. | N.D. |
| 12 | | S. aureus | 390,000 | N.D. | N.D. | N.D. |
| | | A. niger | 190,000 | N.I. | N.I. | N.I. |
| | | C. albicans | 400,000 | N.I. | N.D. | N.D. |
| | B | E. coli | 390,000 | 2.0 | 3.9 | N.D. |
| | | P. aeruginosa | 370,000 | N.D. | N.D. | N.D. |
| | | S. aureus | 180,000 | N.D. | N.D. | N.D. |
| | | A. niger | 300,000 | N.I. | N.I. | N.I. |
| | | C. albicans | 300,000 | N.I. | N.D. | N.D. |
| | C | E. coli | 340,000 | 1.1 | N.D. | N.D. |
| | | P. aeruginosa | 480,000 | N.D. | N.D. | N.D. |
| | | S. aureus | 350,000 | N.D. | N.D. | N.D. |
| | | A. niger | 300,000 | N.I. | N.I. | N.I. |
| | | C. albicans | 430,000 | N.I. | N.I. | N.D. |
| test | A | E. coli | 310,000 | 1.7 | 4.5 | N.D. |
| solution | | P. aeruginosa | 140,000 | N.D. | N.D. | N.D. |
| 13 | | S. aureus | 390,000 | N.D. | N.D. | N.D. |
| | | A. niger | 190,000 | N.I. | N.I. | N.I. |
| | | C. albicans | 400,000 | N.I. | N.D. | N.D. |
| | B | E. coli | 390,000 | 3.0 | N.D. | N.D. |
| | | P. aeruginosa | 370,000 | N.D. | N.D. | N.D. |
| | | S. aureus | 180,000 | N.D. | N.D. | N.D. |
| | | A. niger | 300,000 | N.I. | N.I. | N.I. |
| | | C. albicans | 300,000 | N.I. | N.D. | N.D. |
| | C | E. coli | 340,000 | 0.9 | N.D. | N.D. |
| | | P. aeruginosa | 480,000 | N.D. | N.D. | N.D. |
| | | S. aureus | 350,000 | N.D. | N.D. | N.D. |
| | | A. niger | 300,000 | N.I. | N.I. | N.I. |
| | | C. albicans | 430,000 | N.I. | N.I. | N.D. |
| test | A | E. coli | 310,000 | 1.7 | 4.5 | N.D. |
| solution | | P. aeruginosa | 140,000 | N.D. | N.D. | N.D. |
| 14 | | S. aureus | 390,000 | N.D. | N.D. | N.D. |
| | | A. niger | 190,000 | N.I. | N.I. | N.I. |
| | | C. albicans | 400,000 | N.I. | N.D. | N.D. |
| | B | E. coli | 390,000 | 1.9 | 4.3 | N.D. |
| | | P. aeruginosa | 370,000 | N.D. | N.D. | N.D. |
| | | S. aureus | 180,000 | N.D. | N.D. | N.D. |
| | | A. niger | 300,000 | N.I. | N.I. | N.I. |
| | | C. albicans | 300,000 | N.I. | N.D. | N.D. |
| | C | E. coli | 340,000 | 1.2 | N.D. | N.D. |
| | | P. aeruginosa | 480,000 | N.D. | N.D. | N.D. |
| | | S. aureus | 350,000 | N.D. | N.D. | N.D. |
| | | A. niger | 300,000 | N.I. | N.I. | N.I. |
| | | C. albicans | 430,000 | N.I. | N.I. | N.D. |

As shown in the above results, some test solutions containing 0.1% disodium edetate dehydrate did not meet the criteria in some section, while all test solutions containing 0.05% disodium edetate dehydrate met the criteria in all sections.

### Test Example 5

In order to evaluate the effect of the concentration of isopropyl unoprostone in the solution on the preservative effectiveness test, test solutions shown in tables 8 and 10 were prepared and tested for the preservative effectiveness tests according to the Japanese Pharmacopeia, 15th edition. Sterile test solutions were prepared in the same manner as test solution 1 in test example 1. The test solutions were evaluated under criteria for Category IA product (sterile preparations). Results are summarized in Tables 9 and 11. In the table, “†” indicates the test section that did not meet the criteria.

TABLE 8

| | | | | | pH | |
|---|---|---|---|---|---|---|
| Test | | poly- | disodium | | modi- | pH |
| Solu- | isopropyl | sorbate | edetate | boric | fier | measured |
| tions | unoprostone | 80 | dihydrate | acid | (borax) | value |
| 15 | 0.15% | 1% | 0.05% | 1.71% | 0.04% | 5.99 |
| 16 | 0.15% | 1% | 0.05% | 1.8% | 0.05% | 5.99 |
| 17 | 0.15% | 1% | 0.05% | 1.9% | 0.05% | 6.00 |
| 18 | 0.15% | 1% | 0.02% | 1.71% | 0.02% | 5.98 |
| 19 | 0.15% | 1% | 0.02% | 1.8% | 0.03% | 5.97 |
| 20 | 0.15% | 1% | 0.02% | 1.9% | 0.03% | 5.97 |
| 21 | 0.15% | 1% | 0% | 1.71% | 0.02% | 5.97 |

TABLE 9

| | | | initial | Log reduction | | |
|---|---|---|---|---|---|---|
| | | Micro- | inoculum | 7 | 14 | 28 |
| | facility | organisms | count | days | days | days |
| test | A | E. coli | 250,000 | N.D. | N.D. | N.D. |
| solution 15 | | P. aeruginosa | 200,000 | N.D. | N.D. | N.D. |
| | | S. aureus | 260,000 | N.D. | N.D. | N.D. |
| | | A. niger | 170,000 | N.I. | N.I. | N.I. |
| | | C. albicans | 230,000 | N.I. | N.D. | N.D. |
| test | A | E. coli | 250,000 | 0.5 | 1.7^{†} | 3.9 |
| solution 16 | | P. aeruginosa | 200,000 | N.D. | N.D. | N.D. |
| | | S. aureus | 260,000 | N.D. | N.D. | N.D. |
| | | A. niger | 170,000 | N.I. | N.I. | N.I. |
| | | C. albicans | 230,000 | N.I. | N.D. | N.D. |
| test | A | E. coli | 250,000 | 3.9 | N.D. | N.D. |
| solution 17 | | P. aeruginosa | 200,000 | N.D. | N.D. | N.D. |
| | | S. aureus | 260,000 | N.D. | N.D. | N.D. |
| | | A. niger | 170,000 | N.D. | N.D. | N.D. |
| | | C. albicans | 230,000 | N.I. | N.D. | N.D. |
| test | A | E. coli | 250,000 | 3.0 | N.D. | N.D. |
| solution 18 | | P. aeruginosa | 200,000 | N.D. | N.D. | N.D. |
| | | S. aureus | 260,000 | N.D. | N.D. | N.D. |
| | | A. niger | 170,000 | N.I. | N.I. | N.I. |
| | | C. albicans | 230,000 | N.I. | N.D. | N.D. |
| test | A | E. coli | 250,000 | 4.4 | N.D. | N.D. |
| solution 19 | | P. aeruginosa | 200,000 | N.D. | N.D. | N.D. |
| | | S. aureus | 260,000 | N.D. | N.D. | N.D. |
| | | A. niger | 170,000 | N.I. | N.I. | N.I. |
| | | C. albicans | 230,000 | N.I. | N.D. | N.D. |
| test | A | E. coli | 250,000 | 1.6 | N.D. | N.D. |
| solution 20 | | P. aeruginosa | 200,000 | N.D. | N.D. | N.D. |
| | | S. aureus | 260,000 | N.D. | N.D. | N.D. |
| | | A. niger | 170,000 | N.I. | N.I. | N.I. |
| | | C. albicans | 230,000 | N.I. | N.D. | N.D. |
| test | A | E. coli | 250,000 | 2.7 | N.D. | N.D. |
| solution 21 | | P. aeruginosa | 200,000 | N.D. | N.D. | N.D. |
| | | S. aureus | 260,000 | N.D. | N.D. | N.D. |
| | | A. niger | 170,000 | N.I. | N.I. | N.I. |
| | | C. albicans | 230,000 | N.I. | N.D. | N.D. |

^{†}did not meet the criteria

TABLE 10

| | | | | | pH | |
|---|---|---|---|---|---|---|
| Test | | poly- | disodium | | modi- | pH |
| Solu- | isopropyl | sorbate | edetate | boric | fier | measured |
| tions | unoprostone | 80 | dihydrate | acid | (borax) | value |
| 22 | 0.12% | 1% | 0.05% | 1.71% | 0.04% | 5.99 |
| 23 | 0.12% | 1% | 0.05% | 1.8% | 0.05% | 5.99 |
| 24 | 0.12% | 1% | 0.05% | 1.9% | 0.05% | 6.00 |
| 25 | 0.12% | 1% | 0.02% | 1.71% | 0.02% | 5.98 |
| 26 | 0.12% | 1% | 0.02% | 1.8% | 0.03% | 5.97 |
| 27 | 0.12% | 1% | 0.02% | 1.9% | 0.03% | 5.97 |
| 28 | 0.12% | 1% | 0% | 1.71% | 0.02% | 5.97 |

TABLE 11

| | | | initial | Log reduction | | |
|---|---|---|---|---|---|---|
| | | Micro- | inoculum | 7 | 14 | 28 |
| | facility | organisms | count | days | days | days |
| test | A | E. coli | 250,000 | 0.7 | 2.1^{†} | N.D. |
| solution 22 | | P. aeruginosa | 200,000 | N.D. | N.D. | N.D. |
| | | S. aureus | 260,000 | N.D. | N.D. | N.D. |
| | | A. niger | 170,000 | N.I. | N.I. | N.I. |
| | | C. albicans | 230,000 | N.I. | N.D. | N.D. |
| Test | A | E. coli | 250,000 | 0.4 | 2.0^{†} | N.D. |
| solution 23 | | P. aeruginosa | 200,000 | N.D. | N.D. | N.D. |
| | | S. aureus | 260,000 | N.D. | N.D. | N.D. |
| | | A. niger | 170,000 | N.I. | N.I. | N.I. |
| | | C. albicans | 230,000 | N.I. | N.I. | N.D. |
| Test | A | E. coli | 250,000 | 0.4 | 2.4^{†} | N.D. |
| Solution 24 | | P. aeruginosa | 200,000 | N.D. | N.D. | N.D. |
| | | S. aureus | 260,000 | N.D. | N.D. | N.D. |
| | | A. niger | 170,000 | N.I. | N.I. | N.I. |
| | | C. albicans | 230,000 | N.I. | N.D. | N.D. |
| Test | A | E. coli | 250,000 | N.D. | N.D. | 4.4 |
| Solution 25 | | P. aeruginosa | 200,000 | N.D. | N.D. | N.D. |
| | | S. aureus | 260,000 | N.D. | N.D. | N.D. |
| | | A. niger | 170,000 | N.I. | N.I. | N.I. |
| | | C. albicans | 230,000 | N.I. | N.D. | N.D. |
| Test | A | E. coli | 250,000 | N.D. | N.D. | N.D. |
| Solution 26 | | P. aeruginosa | 200,000 | N.D. | N.D. | N.D. |
| | | S. aureus | 260,000 | N.D. | N.D. | N.D. |
| | | A. niger | 170,000 | N.I. | N.I. | N.I. |
| | | C. albicans | 230,000 | N.I. | N.D. | N.D. |
| Test | A | E. coli | 250,000 | 1.6 | 3.4 | N.D. |
| Solution 27 | | P. aeruginosa | 200,000 | N.D. | N.D. | N.D. |
| | | S. aureus | 260,000 | N.D. | N.D. | N.D. |
| | | A. niger | 170,000 | N.I. | N.I. | N.I. |
| | | C. albicans | 230,000 | N.I. | N.D. | N.D. |
| Test | A | E. coli | 250,000 | 3.5 | N.D. | N.D. |
| Solution 28 | | P. aeruginosa | 200,000 | N.D. | N.D. | N.D. |
| | | S. aureus | 260,000 | N.D. | N.D. | N.D. |
| | | A. niger | 170,000 | N.I. | N.I. | N.I. |
| | | C. albicans | 230,000 | N.I. | N.D. | N.D. |

^{†}did not meet the criteria

Among the test solutions containing 0.05% disodium edetate dehydrate, a higher number of test sections that do not meet the criteria were found in test solutions containing 0.12% isopropyl unoprostone than those in test solutions containing 0.15% isopropyl unoprostone. On the other hand, test solutions containing 0.02% disodium edetate dehydrate, all test solutions tested, i.e. test solutions containing 0.15% or 0.12% isopropyl unoprostone met the criteria in all test sections.

### Test Example 6

Sterile test solutions containing the ingredients shown in Table 12 in water were prepared in the same manner as test solution 1 of test example 1, and were filled aseptically in law-density polyethylene (LDPE) containers respectively. The container was kept at 55° C. for four (4) weeks and the concentration of isopropyl unoprostone in the solution was determined by means of a liquid chromatograph. Results are summarized in Table 12. In the table, represents insufficient stability.

TABLE 12

| ingredients (%) | | | | | | UI concentration (%) | | | |
|---|---|---|---|---|---|---|---|---|---|
| | | | pH | disodium | | vs. indicated, (vs initial) | | | |
| polysorbate | boric | isopropyl | modifier | edetate | | Initial | 55° C. | | test |
| 80 | acid | unoprostone | (borax) | dihydrate | pH | (4° C.) | 2 w | 4 w | solution |
| 1 | 1.71 | 0.12 | 0.018 | 0 | 5.968 | 100.5 | 73.3 | 47.2 | 29 |
| | | | | | | (100) | (72.9)^{‡} | (47.0)^{‡} | |
| | | | 0.024 | 0.02 | 5.953 | 100.5 | 94.8 | 93.8 | 30 |
| | | | | | | (100) | (94.4) | (93.4) | |
| | | | 0.039 | 0.05 | 6.185 | 103.2 | 96.4 | 95.4 | 31 |
| | | | | | | (100) | (93.5) | (92.4) | |
| | | 0.15 | 0.018 | 0 | 5.967 | 97.7 | 78.2 | 57.0 | 32 |
| | | | | | | (100) | (80.0)^{‡} | (58.3)^{‡} | |
| | | | 0.024 | 0.02 | 5.954 | 101.0 | 96.3 | 96.4 | 33 |
| | | | | | | (100) | (95.3) | (95.5) | |
| | | | 0.039 | 0.05 | 6.185 | 100.4 | 95.8 | 95.5 | 34 |
| | | | | | | (100) | (95.4) | (95.1) | |

^{‡}stability was not sufficient

As shown in the above results, disodium edetate dehydrate contribute the stability of isopropyl unoprostone in the test solutions containing 0.12% or 0.15% of isopropyl unoprostone.

### Test Example 7

In order to evaluate the effect of different amount of disodium edetate dehydrate on the stability of isopropyl unoprostone in test solutions containing 0.12% isopropyl unoprostone, test solutions shown in table 13 were prepared and tested for the preservative effectiveness tests according to the Japanese Pharmacopeia, 15th edition. Sterile test solutions were prepared in the same manner as test solution 1 in test example 1. The test solutions were evaluated under criteria for Category IA product (sterile preparations).

In addition, the test solutions 35-38 shown below were aseptically filled in law-dencity polyethylene (LDPE) containers respectively. The container was kept at 55° C. for four (4) weeks and the concentration of isopropyl unoprostone in the solution was determined by means of a liquid chromatograph. Results are summarized in Table 14.

TABLE 13

| | | | | | pH | |
|---|---|---|---|---|---|---|
| Test | | poly- | disodium | | modi- | pH |
| Solu- | isopropyl | sorbate | edetate | boric | fier | measured |
| tions | unoprostone | 80 | dihydrate | acid | (borax) | value |
| 35 | 0.12% | 1% | 0.001% | 1.71% | 0.018% | 5.96 |
| 36 | 0.12% | 1% | 0.002% | 1.71% | 0.018% | 5.96 |
| 37 | 0.12% | 1% | 0.005% | 1.71% | 0.019% | 5.96 |
| 38 | 0.12% | 1% | 0.02% | 1.71% | 0.024% | 6.00 |
| 39 | 0.12% | 1% | 0.03% | 1.71% | 0.022% | 5.96 |

TABLE 14

| | | | | | | Stability |
|---|---|---|---|---|---|---|
| | | | | | | conc of IU |
| | Preservative | | | | | after 4 wks |
| | effectiveness tests | | | | | strage at |
| | | initial | Log reduction | | | 55° C.: |
| | Micro- | inoculum | 7 | 14 | 28 | % vs. |
| | organisms | count | days | days | days | initial conc |
| test | E. coli | 240,000 | 0.4 | 4.2 | N.D. | 92.5 |
| solution | P. aeruginosa | 240,000 | N.D. | N.D. | N.D. | |
| 35 | S. aureus | 410,000 | N.D. | N.D. | N.D. | |
| | A. niger | 220,000 | N.I. | N.I. | N.I. | |
| | C. albicans | 250,000 | N.I. | N.D. | N.D. | |
| test | E. coli | 240,000 | 0.4 | 4.4 | N.D. | 93.8 |
| solution | P. aeruginosa | 240,000 | N.D. | N.D. | N.D. | |
| 36 | S. aureus | 410,000 | N.D. | N.D. | N.D. | |
| | A. niger | 220,000 | N.I. | N.I. | N.I. | |
| | C. albicans | 250,000 | N.I. | N.D. | N.D. | |
| test | E. coli | 240,000 | 0.3 | N.D. | N.D. | 93.2 |
| solution | P. aeruginosa | 240,000 | N.D. | N.D. | N.D. | |
| 37 | S. aureus | 410,000 | N.D. | N.D. | N.D. | |
| | A. niger | 220,000 | N.I. | N.I. | N.I. | |
| | C. albicans | 250,000 | N.I. | N.D. | N.D. | |
| test | E. coli | 240,000 | 0.6 | N.D. | N.D. | 93.3 |
| solution | P. aeruginosa | 240,000 | N.D. | N.D. | N.D. | |
| 38 | S. aureus | 410,000 | N.D. | N.D. | N.D. | |
| | A. niger | 220,000 | N.I. | N.I. | N.I. | |
| | C. albicans | 250,000 | N.I. | N.D. | N.D. | |
| test | E. coli | 240,000 | 0.5 | N.D. | N.D. | No Data |
| solution | P. aeruginosa | 240,000 | N.D. | N.D. | N.D. | |
| 39 | S. aureus | 410,000 | N.D. | N.D. | N.D. | |
| | A. niger | 220,000 | N.I. | N.I. | N.I. | |
| | C. albicans | 250,000 | N.I. | N.D. | N.D. | |

Tests were conducted in Facility A.

As shown in the above results, all test solutions containing 0.001-0.03% disodium edetate dehydrate and 0.12% isopropyl unoprostone met the criteria in all test sections. Enough stability of isopropyl unoprostone were confirmed even in the test solution containing as low as 0.001% disodium edetate dehydrate.

What is claimed is:

1. An aqueous ophthalmic composition comprising:
(a) a fatty acid derivative used in the instant application is represented by the formula (I): 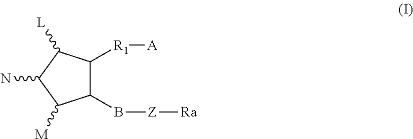
wherein L, M and N are hydrogen, hydroxy, halogen, lower alkyl, hydroxy(lower)alkyl, lower alkanoyloxy or oxo, wherein at least one of L and M is a group other than hydrogen and the five-membered ring may have at least one double bond;
A is —CH₃, —CH₂OH, —COCH₂OH, —COOH or a functional derivative thereof;
B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;
Z is 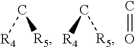
or single bond
wherein, R₄ and R₅ are hydrogen, hydroxy, halogen, lower alkyl, lower alkoxy or hydroxy(lower)alkyl, with the proviso that R₄ and R₅ are not hydroxy and lower alkoxy at the same time;
R₁ is saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and
Ra is saturated or unsaturated lower or medium aliphatic hydrocarbon, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-Oxy group; lower alkoxy; lower alkanoyloxy; cyclo(lower)alkyl cyclo(lower)alkyloxy; aryl; aryloxy; heterocyclic group; or heterocyclic-oxy group, and at least one of carbon atom in the aliphatic hydrocarbon is optionally substituted by oxygen, nitrogen or sulfur;
(b) a polyoxyethylene sorbitan fatty acid ester;
(c) an edetic acid compound
(d) a boric acid and a salt of a boric acid
(e) a pharmaceutically acceptable aqueous carrier, and
(f) no more than 0.005 w/v % of benzalkonium chloride.

2. The composition of claim 1, wherein the amount of benzalkonium chloride in the composition is no more than 0.001 w/v %.

3. The composition of claim 1, which comprises no benzalkonium chloride.

4. The composition of claim 1, which does not comprise a preservative.

5. The composition of claim 1, wherein B is —CH₂—CH₂— and Z is ═O.

6. The composition of claim 1, wherein the fatty acid derivative is isopropyl unoprostone.

7. The composition of claim 1, wherein the polyoxyethylene sorbitan fatty acid ester is Polyoxyethylene sorbitan monooleate.

8. The composition of any one of claim 1, wherein the edetic acid compound is disodium edetate or its hydrate.

9. The composition of claim 1, wherein the boric acid is orthoboric and the salt of a boric acid is borax.

10. The composition of claim 1, wherein the aqueous pharmaceutically acceptable carrier is water.

11. The composition of claim 1, which is formulated as eye drops.

12. The composition of claim 11, which is provided as a sterilized unit dose preparation.

13. The composition of claim 12, which is provided as a daily unit dose preparation.

14. The composition of claim 12, which is provided as a single unit dose preparation.

15. The composition of claim 11, which is provided as a multi dose preparation.

16. The composition of claim 15, which does not comprise a preservative.

17. The composition of claim 1, which comprises in water:
0.15 w/v % or 0.12 w/v % of isopropyl unoprostone;
0.01-0.09 w/v % of disodium edetate dehydrate;
0.8-1.2 w/v % of polysorbate 80;
1.5-2 w/v % of orthoboric acid and
borax in an amount to adjust the pH of the composition to 5.8-6.2.

18. The composition of claim 17, wherein the amount of disodium edetate dehydrate is 0.01-0.03 w/v %.

19. A method for the treatment of a retinal disease or glaucoma and/or ocular hypertension, which comprises administering to a subject in need thereof an aqueous ophthalmic composition of claim 1.
